(19) United States
(12) Patent Application Publication
(10) Pub. No.: US 2026/0311876 A1
(43) Pub. Date: Oct. 8, 2026

(54) AN EARLY WARNING SYSTEM AND METHOD THEREOF

(71) Applicant: TURTLE SHELL TECHNOLOGIES PRIVATE LIMITED, Bengaluru (IN)

(72) Inventors: Gaurav PARCHANI, Bengaluru (IN); Mudit DANDWATE, Bengaluru (IN); Pooja KADAMBI, Bangalore (IN); Ashwathi NAMBIAR, Bangalore (IN); Ishan NIGAM, New Delhi (IN); Sahil SINGH, Gandhinagar (IN)

(21) Appl. No.: 19/481,352

(22) PCT Filed: Nov. 8, 2024
(86) PCT No.: PCT/IN2024/052210

§ 371 Date: Nov. 4, 2025

(30) Foreign Application Priority Data
Mar. 12, 2024 (IN) 202441017806

(51) Int. Cl. G16H 50/20

(52) U.S. Cl. CPC G16H 50/20

(57) ABSTRACT

The present invention discloses an early warning system (100) and method (900) thereof. The system (100) comprises an early warning unit (106) that incorporates a data stream module (202) for collecting at least one input data, a features and transformations module (204) for processing the input data, and a precognitive decision support module (206) for analyzing extracted features or input data to generate personalized early warnings. The invention aims to provide comprehensive health monitoring and predictive analytics, facilitating timely intervention, clinical decision making support and personalized care.

### TECHNICAL FIELD

The field of invention generally relates to healthcare technology. More specifically, it relates to an early warning system and method thereof.

### BACKGROUND ART

The field of healthcare has witnessed a rapid transformation with the advent of advanced technologies, particularly in the domain of health assessment and decision support systems. These technological advancements have opened up new avenues for improving patient care, treatment strategies, and overall healthcare quality. However, despite the progress made, existing systems often fall short of delivering comprehensive and timely insights for healthcare professionals to make informed decisions.

Currently, existing systems do not succeed in providing patient data analysis and care delivery. This fragmentation can result in inefficient workflows, delayed decision-making, and missed opportunities for early intervention.

Other existing systems have tried to address this problem. However, their scope was limited to isolated functionalities, such as data collection or rudimentary analysis Thus, in light of the above discussion, it is implied that there is need for an early warning system and method thereof, which is reliable and does not suffer from the problems discussed above.

### OBJECT OF INVENTION

The principal object of this invention is to provide an early warning system capable of predicting health risks and providing personalized clinical guidance.

Another object of the invention is to enhance the system's ability to integrate various data sources to create a comprehensive representation of patient health, and an ability to collect diverse data types by a data stream module, comprising physiological sensor measurements, historical data, encounter data, and digital health repositories.

Another object of the invention is to implement a feature extraction module to process input data and extract relevant features comprising time-series features, spectral features, statistical features, engineered features, and demographic features, to capture key physiological characteristics and relationships, by using statistical, time-domain, spectral analysis, and transformation techniques.

Another objective of the invention is to extract essential values, trends, and signals from physiological measurements through the vital characteristics module.

Another object of the invention is to utilize extracted features to generate personalized early warnings through a precognitive decision support module.

Another object of the invention is to achieve a personalized medical care module to create a comprehensive and multimodal representation of patient health.

Another object of the invention is to incorporate a collaborative filtering module aimed at constructing a plurality of heterogeneous individualized digital twins, leveraging data shared among similar patients to enhance the accuracy of health monitoring.

Another object of the invention is to provide an intuitive user interface to facilitate secure data input, validation, and visualization of generated care plans and insights for users.

Another object of the invention is to ensure compliance with regulatory requirements and medical ethical standards by providing transparency into the factors influencing system outcomes, facilitating accountability and auditability.

Another object of the invention is to support decision-making by assisting health care professionals or users in interpreting and utilizing the system generated outputs effectively, empowering them to make informed decisions based on system recommendations or predictions.

### BRIEF DESCRIPTION OF DRAWINGS

This invention is illustrated in the accompanying drawings, throughout which, like reference letters indicate corresponding parts in the various figures.

The embodiments herein will be better understood from the following description with reference to the drawings, in which:

FIG. 1 depicts/illustrates a block diagram of an early warning system, in accordance with an embodiment;

FIG. 2 depicts/illustrates a detailed block diagram of an early warning unit, in accordance with an embodiment;

FIG. 3 depicts/illustrates a detailed block diagram of the data stream module, in accordance with an embodiment;

FIG. 4 depicts/illustrates a detailed block a of the features and transformations module, in accordance with an embodiment;

FIG. 5 depicts/illustrates a detailed block diagram of the precognitive decision support module, in accordance with an embodiment;

FIG. 6A depicts/illustrates a graph plotting vital trends for a patient with non-escalated vital signs, in accordance with an embodiment;

FIG. 6B depicts/illustrates a graph of patient with escalated vitals for vital trend-based features, in accordance with an embodiment;

FIG. 6C depicts/illustrates a graph of patient with the weighted sum of features calculated at multiple windows for vital trend-based features with non escalated vitals, in accordance with an embodiment.

FIG. 6D depicts/illustrates a graph of patient with the weighted sum of features calculated at multiple windows for vital trend-based features with escalated vitals, in accordance with an embodiment.

FIG. 6E depicts/illustrates a graph of alert tier for patient with non escalated vitals, in accordance with an embodiment.

FIG. 6F depicts/illustrates a graph of alert tier for patient with escalated vitals, in accordance with an embodiment.

FIG. 7A depicts/illustrates a spectrogram for spectral analysis features for patient with non escalated vitals, in accordance with an embodiment.

FIG. 7B depicts/illustrates a spectrogram for spectral analysis features for patient with escalated vitals, in accordance with an embodiment.

FIG. 7C depicts/illustrates a power spectral density for spectral analysis features for patient with non escalated vitals, in accordance with an embodiment.

FIG. 7D depicts/illustrates a power spectral density for spectral analysis features for patient with escalated vitals, in accordance with an embodiment.

FIG. 7E depicts/illustrates an alert tier for spectral analysis features for patient with non escalated vitals, in accordance with an embodiment.

FIG. 7F depicts/illustrates an alert tier for spectral analysis features for patient with escalated vitals, in accordance with an embodiment.

FIG. 8 depicts/illustrates a user interface for alerts and explainability of alert, in accordance with an embodiment; and

FIG. 9 illustrates a method for an early warning system, in accordance with an embodiment.

### STATEMENT OF INVENTION

The present invention discloses an early warning system comprising a data stream module, a features and transformations module, and a precognitive decision support module. This system aims to predict health risks and offer personalized clinical guidance by integrating various data sources, comprising physiological sensor measurements, historical records, encounter data, and digital health repositories.

A personalized medical care module designed to create a comprehensive and multimodal representation of patient health. This module facilitates informed decision-making by healthcare professionals and contributes to improving patient outcomes by providing tailored medical care insights.

The present invention addresses the need for an advanced early warning system that leverages data-driven approaches to enhance predictive analytics in healthcare, ultimately leading to better patient management and treatment strategies.

### DETAILED DESCRIPTION

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The present invention discloses an early warning system comprising a data stream module, a features and transformations module, and a precognitive decision support module. This system aims to predict health risks and offer personalized clinical guidance by integrating various data sources, comprising physiological sensor measurements, historical records, encounter data, and digital health repositories.

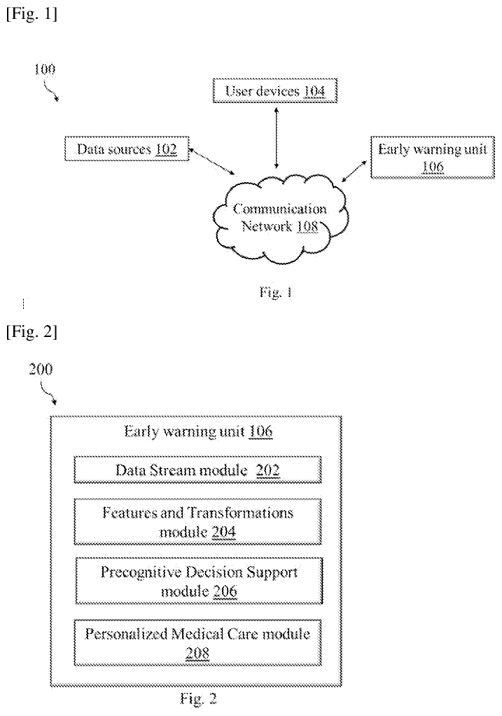

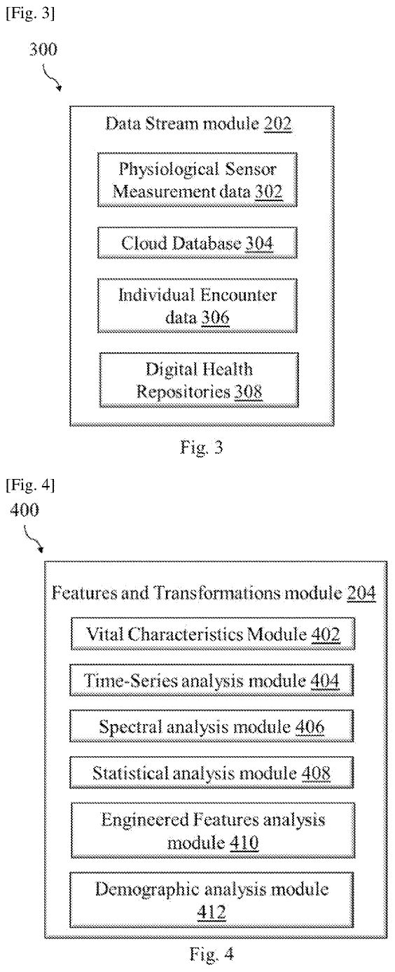

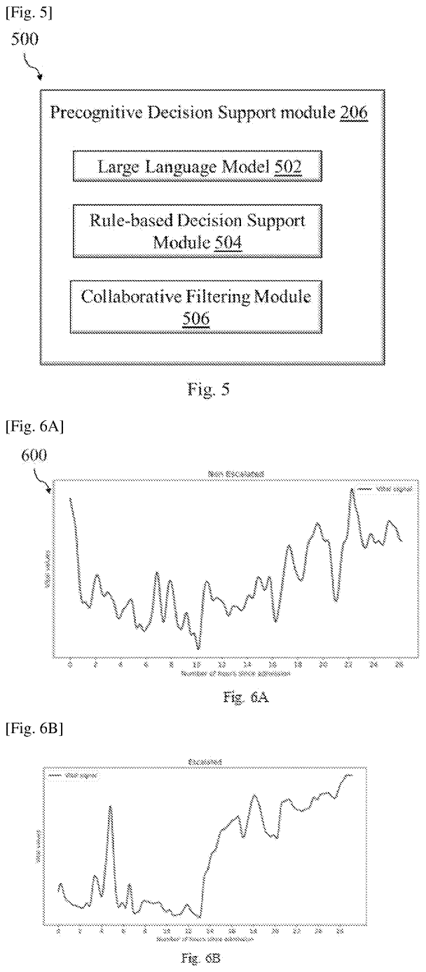

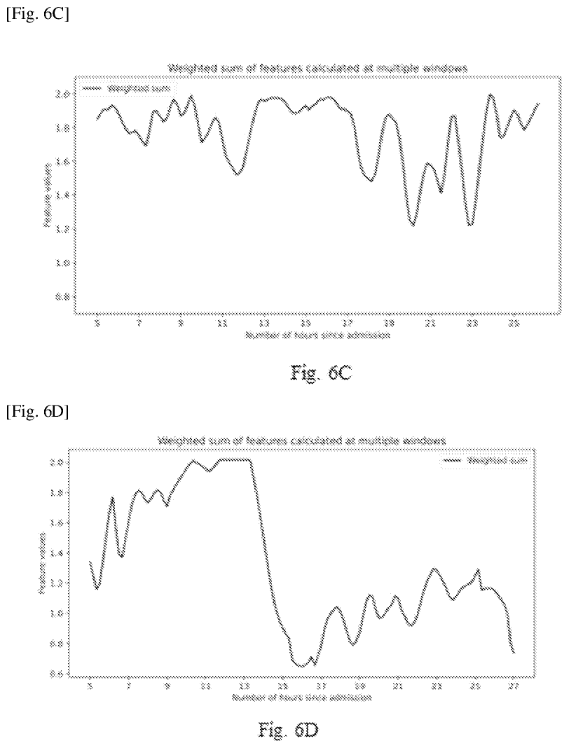

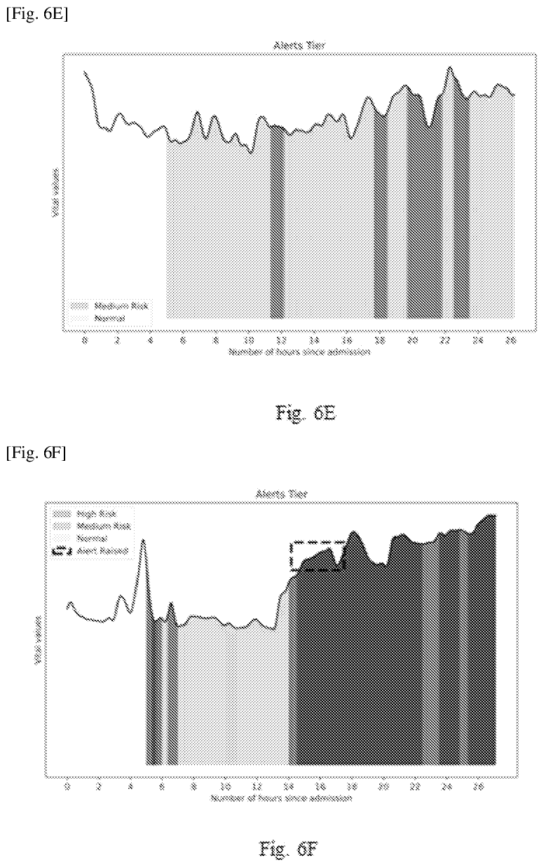

The invention further comprises a personalized medical care module to create a comprehensive and multimodal representation of patient health, facilitating informed decision-making by healthcare professionals and improving patient outcomes.

[FIG. 1] depicts/illustrates a system 100 comprising at least one data source 102, a user device 104, an early warning unit 106 and a communication network 108.

In an embodiment, the data sources 102 encompass various origins or locations from which input data is collected or obtained. The data sources 102 comprise multiple health monitoring devices, cloud databases, historical data and digital health repositories.

In an embodiment, the user device 104, configured to interact with the system 100, providing at least one input data, receiving early warning alerts, and facilitating user interaction with the system's interface, comprises a user application enabling interaction between a suite of LLM and the healthcare professionals, and controls the operations of the system 100. The user devices 102 comprises one or more wearable devices, mobile phones, PDAs, smartphones, smart bands, smartwatches, laptops, computers, etc. In an embodiment the user can be a patient or any other user.

The healthcare professionals are individuals who are trained and licensed to provide medical care and treatment to patients. This term encompasses a wide range of roles and specialties, including doctors, nurses, pharmacists, therapists, technicians, and other allied health professionals.

In an embodiment, a processor comprises the early warning unit 106 is configured to process the collected data, analyze relevant features, and generate personalized early warning alerts based on predictive analytics algorithms. The early warning unit 106, comprises at least one of a computer, laptop, or mobile device.

In an embodiment, the early warning unit 106 incorporates a storage unit which ensures efficient data storage management, and an Input/Output unit which facilitates data input and output, contributing to the overall functionality of the early warning unit 106.

In an embodiment, the communication network 108 is configured to facilitate data exchange between the data sources 102, the user devices 104, and the early warning unit 106, enabling seamless communication and interaction within the system 100.

In an embodiment, the communication network 106 can be a wired or a wireless communication network. The wired communication may comprise LAN, WAN, etc. and wireless communication may comprise cellular networks, WLAN, wireless sensor networks, etc. A set of standard protocols such as, but not limited to, UART, SPI, I2C, Bluetooth, Wi-Fi, LTE, TCP/IP, HTTP, FTP, UDP, IPV4, IPV6, etc. are used by these communication networks to transfer the data between networks and devices.

FIG. [2] depicts/illustrates a detailed block diagram of an early warning unit, comprising a data stream module 202, a features and transformations module 204, a precognitive decision support module 206 and a personalized medical care module 208.

In an embodiment, the data stream module 202 is configured to collect at least one input data from the data sources 102.

In an embodiment, the input data refers to one or more information or raw data that is provided to the system, program, or process as an input for analysis, processing, or manipulation. This data serves as the initial set of information that the system uses to perform its operations or generate outputs.

The input data from the data sources 102 comprises at least one physiological sensor data measurement such as temperature, Ballistocardiography (BCG), Photoplethysmography (PPG), Electrocardiogramd Non-invasive Blood Pressure (NiBP). This measured physiological sensor data serves as a cornerstone for real-time monitoring and analysis within the early warning system 100, for providing essential insights into the patient's health status.

Additionally, the system can utilize supplementary data sources 102 if available, to enhance its efficacy. These additional data sources encompass cloud databases which provide one or more input data comprising at least one of historical data, individual encounter data, and digital health repositories comprising published literature and medical records.

While the physiological sensor measurements input data remains mandatory for system operation, leveraging these supplementary data sources, when accessible, contributes to a more comprehensive understanding of the patient's health profile and can improve the accuracy of early warning alerts and personalized medical care plans generated by the system.

In an embodiment, the historical data refers to past records or information collected over a period of time. It often comprises data points, measurements, or observations recorded at specific intervals or timestamps.

In an embodiment, the individual encounter data typically refers to specific information collected during a single interaction or encounter between a patient and a healthcare provider. This individual encounter data may comprise details such as the patient's symptoms, vital signs, medical history, diagnostic test results, treatments administered, medications prescribed, and any other pertinent observations made during the encounter.

The published literature refers to scholarly works, research articles, books, and other materials that have been formally published and made available to the public. This encompasses a wide range of sources, including academic journals, conference proceedings, textbooks, and online databases, where researchers and experts share their findings, theories, and analyses within various fields of study.

The medical records refer to a comprehensive collection of documentation regarding a patient's health history and care. These records typically comprise information such as medical history, diagnoses, medications, treatment plans, laboratory test results, imaging studies, surgical procedures, and notes from healthcare professionals.

In an embodiment, the features and transformations module 204 is configured for processing the input data also called Raw data or patient data received from the data sources 102. This module employs advanced algorithms and techniques comprising statistical analysis, time-domain analysis, spectral analysis, and various transformations to extract meaningful features from the input data. These features capture key physiological characteristics and trends, providing valuable insights into the patient's health status.

The features and transformations module 204 processes the input data by transforming the input data into a comprehensive set of features, encompassing both basic and higher-order characteristics. Notably, the generation of these features is not isolated; instead, it involves interdependencies where one feature may rely on the computation or analysis of another feature within the features and transformations module 204 itself.

In an embodiment, the precognitive decision support module 206 is configured for analyzing the extracted features and the input data to generate personalized early warnings. Leveraging an ensemble of sophisticated predictive analytics algorithms, this module identifies patterns, trends, and anomalies indicative of potential health risks or adverse events.

The extracted features refer to specific data points or characteristics that have been identified and isolated from a larger dataset through a process of analysis or computation. By applying machine learning and rule-based decision support, the system can proactively alert healthcare professionals to emerging health issues, enabling timely intervention and prevention.

Additionally, the precognitive decision support module 206 incorporates one or more supervised and semi-supervised learning methods by using at least one of a suite of large language model 502, a rule-based decision support module 504, and a collaborative filtering module 506, to analyze the extracted features and the input data. The precognitive decision support module 206 can discern intricate patterns, trends, and anomalies within the input data, enhancing its ability to generate personalized early warnings.

This comprehensive approach enhances the system's predictive capabilities, enabling proactive alerts to healthcare professionals regarding potential health risks or adverse events.

In an embodiment, the personalized medical care module 208 complements the early warning capabilities of the system 100 by offering tailored medical care insights. Drawing upon the comprehensive dataset and analysis performed by the preceding modules, this module generates holistic and robust representations of patient health.

Healthcare professionals can utilize these insights to formulate personalized care plans, optimize treatment strategies, and enhance patient outcomes.

FIG. [3] depicts/illustrates a detailed block diagram of the data stream module 202, comprising a physiological sensor measurement data 302, a cloud database 304, an individual encounter data 306 and a digital health repositories 308.

In an embodiment, the physiological sensor measurement data 302 represents real-time data obtained from a variety of sensors monitoring vital signs such as temperature, Ballistocardiography (BCG), Photoplethysmography (PPG), Electrocardiogramd Non-invasive Blood Pressure (NiBP). Advantageously, these measurements offer valuable insights into the patient's physiological status and enable continuous monitoring of key health indicators.

In an embodiment, the cloud database 304 contains a wealth of historical data, both at the individual patient level and aggregated population data. By accessing this repository, the system gains access to longitudinal patient records, past medical history, treatment outcomes, and demographic information. This historical context enhances the system's ability to detect trends, identify risk factors, and personalize early warning alerts based on individual patient profiles.

In an embodiment, the individual encounter data 306 captures information gathered during specific patient encounters, such as clinical visits, hospital admissions, or telehealth consultations. This data may comprise environmental factors like room air quality, subjective indicators such as pain index, and observations like changes in skin color. By incorporating encounter-specific data, the system can contextualize the patient's health status and provide targeted interventions based on immediate needs.

In an embodiment, the digital health repositories 308 serve as repositories of external data sources, comprising peer-reviewed literature and medical repositories. These repositories contain a vast array of medical knowledge, research findings, treatment guidelines, and clinical best practices. By accessing this external knowledge base, the system 100 can stay updated with the latest advancements in medical science, leverage evidence-based practices, and enhance the accuracy and efficacy of its early warning capabilities.

FIG. [4] depicts/illustrates a detailed block diagram of features and transformations module 204, comprising a vital characteristics module 402, a time-series analysis module 404, a spectral analysis module 406, a statistical analysis module 408, an engineered feature analysis module 410 and a demographic analysis module 412.

The features and transformations module 204 is designed to meticulously analyze input data, extracting multiple features crucial for health assessment. These features encompass various dimensions of physiological dynamics, comprising at least one of time-series features such as heart rate variability and respiratory rate patterns, spectral features derived from frequency domain analysis of parameters like ECG and PPG, statistical features like mean and standard deviation capturing data distribution characteristics, engineered features crafted to encapsulate nuanced physiological relationships comprising short term and long term changes (trends in values and changes in signal morphology and features), and demographic features such as age, gender, and ethnicity, providing a holistic understanding of the patient's health profile.

In an embodiment, the vital characteristics module 402 focuses on extracting essential values from physiological measurements, such as heart rate variability, respiratory rate patterns, and blood pressure trends.

In an embodiment, the time-series analysis module 404 is specialized in analyzing and identifying temporal patterns in vital values, necessitating individual historical records for comprehensive assessment. The time-series analysis module 404 uses at least one of recurrence plots, Poincare plots, and similar analyses. The time-series analysis module 404 is configured to examine various trends comprising at least one of heart rate variability, respiratory rate patterns, and trends in vital values over time. Additionally, the module integrates the concept of analyzing weighted sums of vital values and trends over different time periods to discriminate between patients likely to decompensate in the future and those who are not.

In an embodiment, the spectral analysis module 406 conducts frequency domain analysis on physiological vitals, trends, and signals, such as ECG, PPG, or QRS complex parameters, to extract spectral features like P-wave amplitude, PPG pulse wave amplitude, and rise time. These spectral features provide valuable insights into the underlying physiological characteristics of the patient, contributing to the system's ability to discern nuanced health indicators and differentiate between patients requiring acute care. The spectral analysis module 406 uses Fourier transforms, wavelet transforms, and similar signal transformations.

In an embodiment, the statistical analysis module 408 performs statistical computations on various measured parameters, comprising mean, standard deviation, skewness, and kurtosis, to derive statistical features relevant to patient health.

In an embodiment, the engineered features analysis module 410 is designed to offer at least one of signal morphology analysis, combinations and transformations of basic features to capture specific physiological relationships effectively.

In an embodiment, the demographic analysis module 412 determines demographic features such as age, gender, and ethnicity, which contribute to a comprehensive understanding of the patient's health profile.

In an embodiment, the features and transformations module 204 enhances explainability of the system's process and outputs by providing transparent insights and to improve an interpretability of the system's outputs, making it easier for healthcare professionals to understand the generated insights. The features and transformations module 204 also incorporates linear data transformations to enhance the processing of the input data. The linear data transformations use Principal Component Analysis (PCA), Independent Component Analysis (ICA), and Similar Analysis. These transformations are strategically employed within the module's constituent modules, depending on the system requirements of each analysis. For example, in the time-series analysis module 404, linear transformations like scaling or normalization are utilized to standardize various time-series features, ensuring their comparability across the dataset. Similarly, within the statistical analysis module 408, linear transformations such as computing the mean or standard deviation entail linear operations on the data, aiding in extracting essential statistical insights.

FIG. [5] depicts/illustrates a detailed block diagram of the precognitive decision support module 206, comprising a suite of large language model 502, a rule-based decision support module 504, and a collaborative filtering module 506.

In the early warning system 100, the rule-based decision support module 504 serves as a fundamental component, essential for the operation of the precognitive decision support module 206. The rule-based decision support module is mandatory, and the inclusion of additional modules such as a suite of Large Language Models 502 and a collaborative filtering module 506 is optional. These supplementary modules offer advanced capabilities for analyzing extracted features and generating comprehensive diagnostic options, as well as building individualized heterogenous digital twins for enhanced health monitoring accuracy, respectively. Their inclusion enhances the system's ability to provide nuanced insights and recommendations to healthcare professionals but is not strictly necessary for its basic operation.

In an embodiment, the Large Language Models (LLMs) 502 configured to analyze extracted features, employs advanced natural language processing techniques to comprehensively access the extracted features and generate at least one comprehensive diagnostic option and providing clinical context for assisting healthcare professionals in their decision making process. The LLMs are engineered using diverse chain of thought prompting methods specifically designed to elicit a comprehensive collection of advice for prognostics and differential diagnoses. By prompting various lines of thinking, the LLMs facilitate informed clinical judgments by providing a broad spectrum of potential diagnoses and treatment recommendations tailored to the patient's condition. It assists healthcare professionals by generating a detailed diagnostic options based on the analyzed features, enhancing the decision-making process by providing a wide range of potential diagnoses and treatment recommendations. The LLMs prompt is engineered using diverse chain-of-thought prompting methods that are designed to elicit a diverse collection of advice for prognostics/differential diagnoses.

In addition to analyzing extracted features and providing diagnostic options, the suite of Large Language Models (LLMs) 502 within the system 100 undergoes a rigorous fine-tuning process. This involves training the LLMs on data sourced from the digital health repositories 308. This augmentation enables the LLMs to collaboratively provide Deliberative Differential Diagnoses to the healthcare professionals.

In an embodiment, the rule-based decision support module 504 utilizes predefined rules and algorithms to analyze extracted features and individual historical data, calculating personalized risk indices and generating at least one early warning alerts based on predetermined criteria. The at least one early warning alerts are distributed based on at least one of number, tier and relative timing, in order to represent patient improvement or deterioration. The frequency and distribution of alerts within specific time intervals, such as 3-hour, 6-hour, or 24-hour windows, with rolling windows in steps of 30 minutes or 1 hour, as determined by the system's algorithms. The number, tier, and pattern of alerts within these time windows serve as crucial indicators of patient improvement or deterioration. For example, a higher frequency of alerts within a shorter time period might signify rapid deterioration, while a consistent pattern of alerts over longer intervals could indicate chronic or stable conditions. These alert distributions play a significant role in healthcare professionals decision-making processes, aiding in patient triage, risk assessment, and the weighting of other features extracted by the system. In an embodiment the frequency of the warning alert can be changed dynamically. The number of alerts (frequency) and the change in distribution over time itself is a feature representing the patient's state.

$$Y = β 0 + f 1 ( x 1 ) + f 2 ( x 2 ) + … + fn ( xn )$$

The varied inputs (xi) from the features and transformations module 204 along with a collection of transformations functions (ﬁ) customized based upon the cloud database are used in a supervised learning regime to learn a personalized risk indexation for the patient. A baseline score (β0) is assigned based on the level of acute care required by the patient. The output of this system (Y) serves as an interpretable and explainable risk indexation for the system

In an embodiment, the collaborative filtering module 506 operates within the early warning system to enhance its predictive capabilities. It accomplishes this by dynamically assessing and leveraging data shared among patients with similar medical profiles. By analyzing patterns and correlations in shared data, the module determines the relevance of this information to individual patients. Using advanced algorithms, it constructs or builds a plurality of heterogenous individualized digital twins, which are comprehensive representations of each patient's health status and risks. These digital twins enable more accurate health monitoring and prediction by capturing personalized factors and nuances specific to each patient. The collaborative filtering module 506 improves the system's 100 ability to deliver personalized healthcare interventions and anticipate medical needs effectively.

The digital twins represent comprehensive snapshots of individual patients' health profiles, incorporating diverse data sources and risk factors. By dynamically assessing and integrating data shared among patients with similar medical backgrounds, the module constructs these personalized digital twins. Each twin encapsulates unique patient-specific factors and nuances, contributing to a more accurate understanding of individual health statuses and risks. Advantageously, this approach enables the system to deliver tailored healthcare interventions and anticipate medical needs more effectively, ultimately improving patient outcomes and healthcare delivery efficiency.

The system also implements a novel approach to train the collaborative filtering module 506, combining unsupervised and supervised learning algorithms with population records. This results in a hybrid digital twin representation for each patient, capturing diverse data streams and risk factors. These digital twins serve as dynamic tools for health monitoring, continuously updated with new insights. By incorporating multiple matches, the system assigns higher weights to twin fragments, enhancing its accuracy in predicting patient health status and risks.

In an embodiment, the personalized medical care module 208 integrates with the decision support module 206 to create a comprehensive and multimodal representation of patient health based on the analyzed features and generated insights. This module ensures that interventions and care plans are tailored to individual patient needs, optimizing patient outcomes and enhancing the quality of care provided.

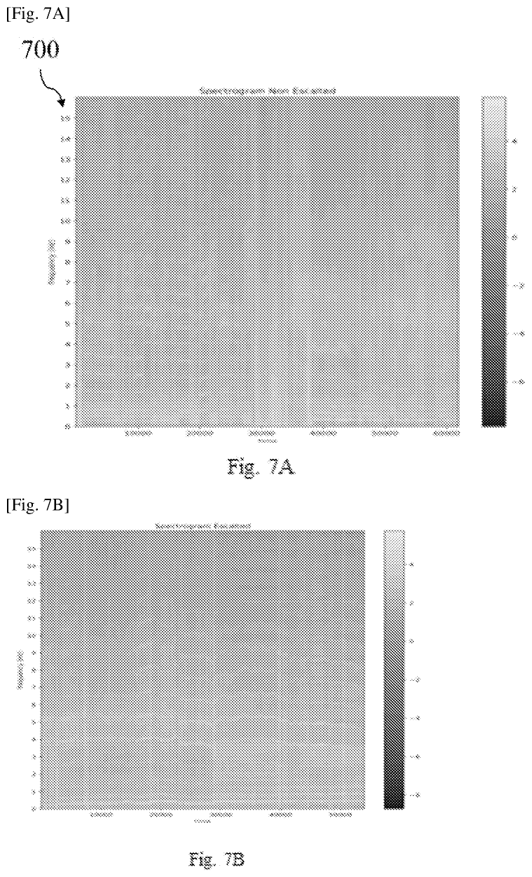

The statistics derived from the data processed by the system described herein serve as valuable inputs for chain-of-thought prompting. This mechanism enables higher-level reasoning, potentially leading to the formulation of comprehensive and differential diagnoses. By integrating statistical insights from the EWS data, healthcare professionals can engage in more nuanced and informed decision-making processes, facilitating the identification of various medical conditions and tailored treatment strategies. The system leverages insights derived from processed data to allow the generation of diverse prompts for multiple specialized AI agents to provide diverse perspectives that facilitate a nuanced and informed decision-making process for healthcare professionals. The formulation of diverse and comprehensive advice generated by the suite of AI agents aids in improved differential diagnoses. This collaborative framework enables healthcare professionals to identify various medical conditions and devise tailored treatment strategies effectively.

[FIG. 6A] depicts/illustrates a graph plotting vital trends for a patient with non-escalated vital signs.

The graph depicts vital values of the patient plotted against the number of hours since their admission into the health care institution.

[FIG. 6B] depicts/illustrates a graph of patient with escalated vitals for vital trend-based features, in accordance with an embodiment.

The graph depicts vital signals of the patient plotted against the number of hours since their admission into the health care institution.

[FIG. 6C] depicts/illustrates a graph of patient with the weighted sum of features calculated at multiple windows for vital trend-based features with non escalated vitals, in accordance with an embodiment.

The graph depicts a feature values of the patient plotted against the number of hours since their admission into the health care institution.

[FIG. 6D] depicts/illustrates a graph of patient with the weighted sum of features calculated at multiple windows for vital trend-based features with escalated vitals, in accordance with an embodiment.

The graph depicts feature values of the patient plotted against the number of hours since their admission into the health care institution.

[FIG. 6E] depicts/illustrates a graph of alert tires for patient with non escalated vitals, in accordance with an embodiment.

The graph depicts vital values of the patient plotted with normal and medium risk against the number of hours since their admission into the health care institution.

[FIG. 6F] depicts/illustrates a graph of alert tires for patient with escalated vitals, in accordance with an embodiment.

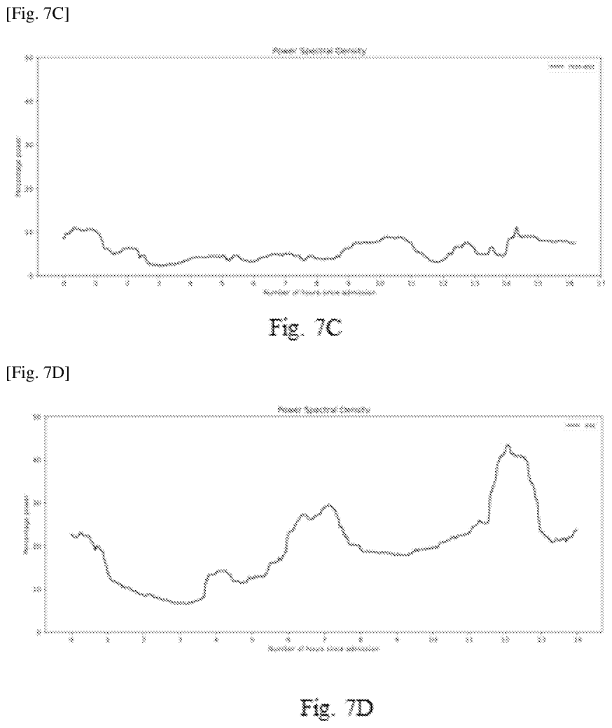

The graph depicts vital signals of the patient plotted with normal, medium, high risk and alert raised against the number of hours since their admission into the health care institution.

[FIG. 7A] depicts/illustrates a spectrogram non-escalated for spectral analysis features for patient with non escalated vitals, in accordance with an embodiment.

The spectrogram graphically represents the distribution of signal power across different frequency bands, providing valuable information for assessing the patient's physiological dynamics and health status.

[FIG. 7B] depicts/illustrates a spectrogram escalated for spectral analysis features for patient with escalated vitals, in accordance with an embodiment.

The spectrogram graphically represents the distribution of signal power across different frequency bands, providing valuable information for assessing the patient's physiological dynamics and health status.

[FIG. 7C] depicts/illustrates a power spectral density for spectral analysis features for patient with non escalated vitals, in accordance with an embodiment.

The graph depicts percentage power of the patient plotted against the number of hours since their admission into the health care institution.

[FIG. 7D] depicts/illustrates a power spectral density for spectral analysis features for patient with escalated vitals, in accordance with an embodiment.

The graph depicts percentage power of the patient plotted against the number of hours since their admission into the health care institution.

[FIG. 7E] depicts/illustrates an alert tier for spectral analysis features for patient with non escalated vitals, in accordance with an embodiment.

The graph depicts percentage power of the patient plotted with normal, medium, high risk and alert raised against the number of hours since their admission into the health care institution.

[FIG. 7F] depicts/illustrates an alert tier for spectral analysis features for patient with escalated vitals, in accordance with an embodiment.

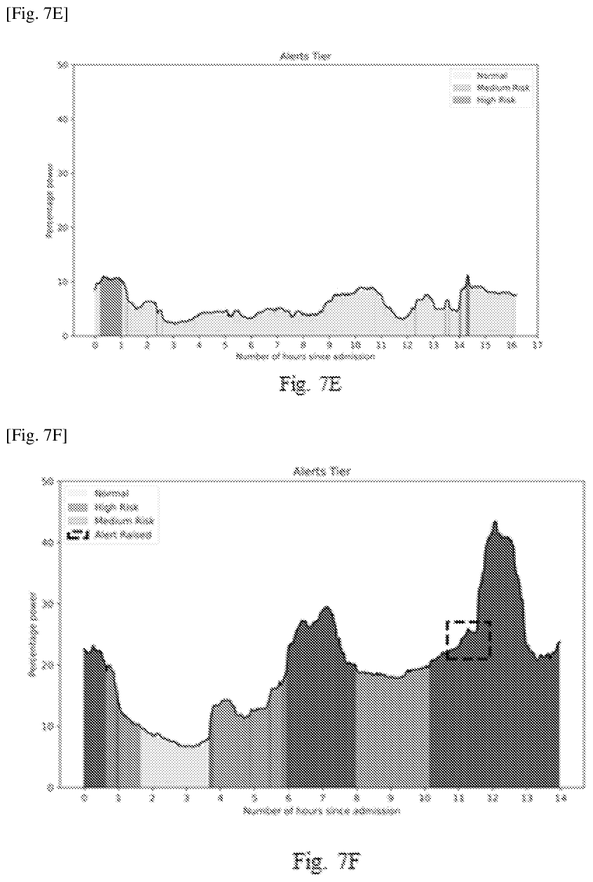

The graph depicts percentage power of the patient plotted with normal, medium, high risk and alert raised against the number of hours since their admission into the health care institution.

[FIG. 8] depicts/illustrates a user interface for alerts and explainability of alert, in accordance with an embodiment.

[FIG. 8] showcases a user interface that refers to the point of interaction between a user and a user device or software application. It is designed to manage alerts and provide comprehensive explainability for each alert generated by the system.

In an embodiment, the explainability refers to the ability of the system to provide clear and understandable explanations for its decisions or outputs. For example, consider a scenario where a patient's vital signs exhibit fluctuations over time, and healthcare professionals need to understand the underlying factors contributing to these changes. The features and transformations module analyzes the patient's historical data, extracting multiple features such as heart rate variability, respiratory rate patterns, and trends in vital values. By presenting these extracted features in a transparent manner, along with explanations of how each feature is derived and its significance, the module provides healthcare professionals with insights into the factors influencing the patient's current health status.

The module may highlight a significant increase in heart rate variability over the past week, indicating potential physiological stress or instability. By transparently presenting this information and explaining the relationship between heart rate variability and stress responses, healthcare professionals gain a clearer understanding of the patient's condition and can make informed decisions regarding further evaluation or intervention.

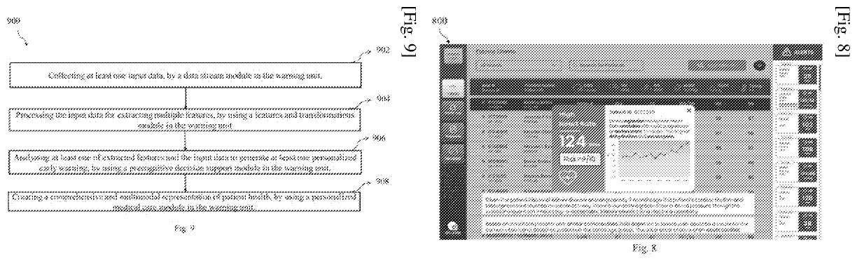

The features and transformations module enhances the explainability of the system by providing healthcare professionals with transparent insights into the extracted features, thereby facilitating a deeper understanding of the patient's health status and supporting more effective healthcare professionals decision-making

[FIG. 9] illustrates a method 900 for an early warning system. The method begins with collecting at least one input data, by a data stream module in the early warning unit, by at least one data source, as depicted at step 902. Subsequently, the method 900 discloses processing the input data for extracting multiple features, by using a features and transformations module in the early warning unit, as depicted at step 904. Thereafter, the method 900 discloses analyzing at least one of extracted features and the input data to generate at least one personalized early warning, by using a precognitive decision support module in the early warning unit, as depicted at step 906. Thereafter, the method 900 discloses creating a comprehensive and multimodal representation of patient health, by using a personalized medical care module in the early warning unit, as depicted at step 908.

The advantages of the current invention include:
Improved explainability: The explainability feature of the system fosters enhanced understanding among healthcare professionals by providing insights into the reasoning behind its outputs or alerts. Explainability in this context refers to the clinical and biological basis for the early warning predictions and assessments.
Explainability of alert: Explainability helps build trust in systems by providing clear and understandable insights into how decisions are made. When users can understand the reasoning behind system generated outcomes, they are more likely to trust and accept the system's recommendations or predictions.
Vital characteristics module: the system utilizes the vital characteristics module to extract essential values, trends, and signals from physiological measurements, for enhancing the system's ability to capture key physiological characteristics effectively.
Comprehensive Data Collection: The system systematically collects diverse input data from various sources, comprising hardware devices, electronic health records (EHRs), and manual inputs by healthcare professionals, ensuring a comprehensive and holistic view of the patient's health.
Personalized Care: By analyzing a patient's historical data, trends, and current condition, the system can generate personalized care plans tailored to individual patient needs, resulting in improved patient outcomes.
Real-time Insights: The system utilizes advanced AI and machine learning techniques to provide real-time insights, enabling healthcare professionals to make informed decisions promptly, potentially saving lives in critical situations.
Risk Stratification: The inclusion of a risk stratification module enables healthcare professionals to identify patients at higher risk of specific health issues or complications, facilitating timely intervention and preventive measures.
Enhanced Decision Support: Healthcare professionals have access to a wealth of data, published research and data from other users with similar comorbidities, empowering them with evidence-based information for making informed decisions.
Efficient Data Management: The system's storage unit ensures efficient data management, enabling the secure storage and retrieval of patient data, and supporting continuity of care and research.
User-Friendly Interface: The user interface module provides a user-friendly platform for healthcare professionals, streamlining data input, validation, and the presentation of care plans and insights, enhancing usability and efficiency.
Collaborative Insights: By comparing data from users with similar health conditions, the system fosters collaborative insights and shared knowledge, benefiting both healthcare professionals and patients.
Data Exchange: The communication network facilitates seamless data exchange between the system, healthcare professionals, hardware devices, EHRs, and external databases, ensuring up-to-date information and efficient data acquisition.
Adaptability: The system can adapt to various healthcare settings and domains, making it a versatile tool for healthcare professionals across different specialties and facilities.

Applications of the current invention include:
Clinical Healthcare Settings: This system can be implemented in hospitals, clinics, and other healthcare professionals to assist healthcare professionals in diagnosing and treating patients. It provides valuable insights, personalized care plans, and risk assessments, enhancing the quality, resource utilization and efficiency of patient care.
Emergency Medicine: In emergency departments, the system's real-time insights and risk stratification capabilities can aid in prioritizing and treating patients based on their critical conditions.
Chronic Disease Management: Patients with chronic illnesses can benefit from continuous monitoring and personalized care plans, improving their management of conditions like diabetes, hypertension, and heart disease.
Telemedicine: The system supports remote patient monitoring, making it ideal for telemedicine applications. Healthcare providers can monitor patients' health remotely and intervene when necessary.
Medical Research: Researchers can use the system to access a wealth of patient data, published research and data from patients with similar comorbidities. This can facilitate clinical trials, medical studies, and data-driven research.
Health Insurance and Payers: Health insurance companies and payers can use the system to assess risk factors and determine appropriate coverage and pricing for policyholders.
Pharmaceutical Industry: The system can assist in clinical trials, drug development, and pharmacovigilance by providing comprehensive patient data and insights into drug efficacy and safety.
Public Health: Public health agencies can utilize the system for disease surveillance, outbreak detection, and monitoring population health trends.
Fitness and Wellness: The system can be adapted for consumer wellness applications, providing individuals with insights into their health and fitness, and promoting healthier lifestyles.
Elderly Care: In assisted living facilities and elderly care settings, the system can monitor the health of elderly residents and provide early warnings of health issues.
Mental Health: The system can incorporate data related to mental health conditions and provide insights and interventions for mental health care providers and patients.
Sports Medicine: In sports medicine, the system can monitor athletes' health and performance, helping coaches and trainers optimize training regimens and prevent injuries.
Occupational Health: Companies can use the system to monitor the health and safety of their employees, especially in physically demanding or high-risk industries.
Government Healthcare Initiatives: Governments can employ the system as part of national healthcare initiatives to improve the overall health of their populations and reduce healthcare costs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described here.

1. An early warning system (100), comprising:
a processor comprises an early warning unit (106) comprises:
a data stream module (202) configured to collect at least one input data;
a features and transformations module (204) configured to process the input data to extract multiple features;
a precognitive decision support module (206) configured to analyze at least one of extracted features and the input data to generate at least one personalized early warning; and
a personalized medical care module (208) configured to represent a comprehensive and multimodal representation of patient health.

2. The system (100) as claimed in claim 1, wherein the data stream module (202) collects the input data comprising:
a physiological sensor measurement data (302) comprising at least one of temperature, Ballistocardiography (BCG), Photoplethysmography (PPG), Electrocardiogramand Non-invasive Blood Pressure (NiBP);

3. The system (100) as claimed in claim 2, wherein the data stream module (202) can collect data comprising at least one of:
a cloud database (304) comprising at least one of an individual historical data, and population historical data;
an individual encounter data (306) comprising at least one of room air, pain index, and skin color; and
a digital health repositories (308) comprising at least one of peer-reviewed published literature, and medical repositories.

4. The system (100) as claimed in claim 1, wherein the multiple features comprise at least one of:
time-series features comprising at least one of heart rate variability, respiratory rate patterns, trends in vital values;
spectral features comprising frequency domain analysis of at least one of ECG, PPG, or QRS complex parameters, P-wave amplitude, PPG pulse wave amplitude and rise time;
statistical features comprising at least one of mean, standard deviation, skewness, kurtosis of various measured parameters;
engineered features are combinations or transformations of basic features to capture specific physiological relationships; and
demographic features comprising at least one of age, gender and ethnicity.

5. The system (100) as claimed in claim 1, wherein the features and transformations module (204) comprises at least one of:
a vital characteristics module (402) configured to extract at least one values to capture key physiological characteristics, comprises at least one of heart rate variability, respiratory rate patterns, blood pressure trends, and temperature fluctuations:
a vital characteristics module (402) configured to extract at least one values to capture key physiological characteristics, comprises at least one of heart rate variability, respiratory rate patterns, blood pressure trends, and temperature fluctuations.
a time-series analysis module (404) configured to analyze and determine at least one trend comprising at least one of heart rate variability, respiratory rate patterns, trends in vital values;
a spectral analysis module (406) configured to conduct a frequency domain analysis of ECG, PPG, or QRS complex parameters, P-wave amplitude, PPG pulse wave amplitude and rise time;
a statistical analysis module (408) configured to analyze mean standard deviation, skewness, kurtosis of various measured parameters;
an engineered features analysis module (410) configured to provide at least one of signal morphology, combinations, and transformations of basic features to capture specific physiological relationships; and
a demographic analysis module (412) configured to determine age, gender and ethnicity.

6. The system (100) as claimed in claim 1, wherein the precognitive decision support module (206) comprises:
a rule based decision support module (504) configured to analyze extracted features to calculate a personalized risk index and generate at least one early warning alert.

7. The system (100) as claimed in claim 1, wherein the precognitive decision support module (206) comprises at least one of:
a suite of Large Language Models (502) configured to analyze extracted features, facilitate chain-of-thought prompting, and generate at least one comprehensive diagnostic option and providing clinical context for assisting healthcare professionals in their decision making process;
a collaborative filtering module (506) configured to build a plurality of heterogenous individualized digital twins for each patient by dynamically weighing data shared with similar patients, for health monitoring accuracy.

8. The system (100) as claimed in claim 6, wherein the at least one early warning alerts are distributed based on at least one of number, frequency, tier and relative timing, in order to represent patient state.

9. The system (100) as claimed in claim 1, comprising at least one user device (104) configured to provide a user application to facilitate secure data input and validation, and visualize and present generated care plans and insights to user, wherein the user application enables interaction between a suite of Large Language Models and the healthcare professional.

10. A method (900) for an early warning system (100), comprising:
collecting at least one input data, by a data stream module (202) in the early warning unit (106);
processing the input data for extracting multiple features, by using a features and transformations module (204) in the early warning unit (106);
analyzing at least one of extracted features and the input data to generate at least one personalized early warning, by using a precognitive decision support module (206) in the early warning unit (106); and
creating a comprehensive and multimodal representation of patient health, by using a personalized medical care module (208) in the early warning unit (106).

11. The method (900) as claimed in claim 10, comprising configuring the features and transformations module (204) comprising:
extracting at least one values to capture key physiological characteristics, comprises at least one of heart rate variability, respiratory rate patterns, blood pressure trends, and temperature fluctuations, by using a vital characteristics module (402);
analyzing and determining at least one trend comprises at least one of heart rate variability, respiratory rate patterns, trends in vital values, by using a time-series analysis module (404);
conducting a frequency domain analysis of ECG, PPG, or QRS complex parameters, P-wave amplitude, PPG pulse wave amplitude and rise time, by using a spectral analysis module (406);
analyzing mean, standard deviation, skewness, kurtosis of various measured parameters, by using a statistical analysis module (408);
providing at least one of signal morphology, combinations, and transformations of basic features to capture specific physiological relationships, by using a engineered features analysis module (410); and
determining age, gender and ethnicity, by using a demographic analysis module (412).

12. The method (900) as claimed in claim 10, comprising configuring the precognitive decision support module (206) comprises:
analyzing extracted features to calculate a personalized risk index and generate at least one early warning alert, by using a rule based decision support module (504).

13. The method (900) as claimed in claim 10, comprising configuring the precognitive decision support module (206) comprises:
analyzing extracted features, facilitating chain-of-thought prompting, and generating at least one comprehensive diagnostic option and providing clinical context for assisting healthcare professionals in their decision making process, by using a suite of Large Language Models (502); and
building a plurality of heterogeneous individualized digital twins for each patient by dynamically weighing data shared with similar patients, for health monitoring accuracy, by using a collaborative filtering module (506).

14. The method (900) as claimed in claim 12, distributing the at least one early warning alerts based on at least one of number, tier and relative timing, in order to represent patient improvement or deterioration.

15. The method (900) as claimed in claim 10, comprising:
providing a user application, to facilitate secure data input and validation, and visualize and present generated care plans and insights to users, by using at least one user device (104); and
enabling interaction between a suite of Large Language Models and the healthcare professional by the user application.
